(19) United States
(12) Patent Application Publication
(10) Pub. No.: US 2025/0170750 A1
(43) Pub. Date: May 29, 2025

(54) SELECTIVE ADJUSTMENT OF HARDNESS AND TRANSLUCENCY IN CERAMIC MULTI-COLOR INKJET 3D PRINTING

(71) Applicant: Ivoclar Vivadent AG, Schaan (LI)

(72) Inventors: Hendrik JOHN, Buchs (CH); Frank ROTHBRUST, Röns (AT); Sebastian KROLIKOWSKI, Richterswil (CH); Christian RITZBERGER, Grabs (CH)

(21) Appl. No.: 18/952,292

(22) Filed: Nov. 19, 2024

(30) Foreign Application Priority Data
Nov. 29, 2023 (EP) 23213084.9

(51) Int. Cl. B28B 1/00; A61C 13/00; B33Y 10/00; B33Y 30/00; B33Y 70/10

(52) U.S. Cl. CPC B28B 1/001; A61C 13/0013; B28B 1/008; B33Y 10/00; B33Y 30/00; B33Y 70/10

(57) ABSTRACT

A method of producing a dental restoration by jet-printing, comprising the steps of jet-printing (S101) one or more layers of the dental restoration by means of a ceramic slurry; and jet-printing (S102) a translucency enhancer or opacity enhancer onto the one or more layers.

### CROSS-REFERENCE TO RELATED APPLICATIONS

This application claims priority to European Patent Application No. 23213084.9 filed on Nov. 29, 2023, which is incorporated herein by reference in its entirety.

### TECHNICAL FIELD

The present invention relates to a method of producing a dental restoration by jet-printing and to a production apparatus for producing a dental restoration by jet-printing.

### BACKGROUND

It is possible to process aqueous or solvent-based ZrO₂ slurries into dental restorations layer by layer by means of jet-printing of material (also known as the inkjet process). Jet-printing makes it possible to apply different slurries in a location-selective manner. These slurries can be differently pre-colored and in addition may have different translucencies and/or different mechanical properties.

In the case of ZrO₂ slurries, different yttrium-doped ceramic powders can be used for the different strengths. The yttrium doping is also responsible for the degree of translucency. The different yttrium-doped ceramic powders have different properties. For example, three different starting powders or slurries can be used to represent three levels of translucency.

However, if different pre-colored slurries are used, all of which also have different translucency and/or different mechanical properties, each of these slurries is assigned its own print head. If, for example, a general CYMK color scheme is used to cover the entire color space, 3×4=12 slurries must be provided in 12 print heads. The production of these different slurries is also complex. Due to this large number of slurries, the provision and processing effort is correspondingly high.

US 20200017699, 20120308837 and 20100249305 are directed to 3D printing and are hereby incorporated by reference in their entirety.

### SUMMARY

It is the technical task of the present invention to build up a dental restoration with only a few base slurries, at best a single one, and to selectively adjust the opacity or translucency locally when producing a dental restoration.

This task is solved by subject-matter according to the independent claims. Technically advantageous embodiments are the subject-matter of the dependent claims, the description and the drawings.

According to a first aspect, the technical task is solved by a method of producing a dental restoration by jet-printing, comprising the steps of jet-printing of one or more layers of the dental restoration by means of a ceramic slurry; and jet-printing of a translucency enhancer or opacity enhancer onto the one or more layers. In jet-printing, the respective materials are ejected and applied location-selectively drop by drop by a movable print head (inkjet process).

A translucency enhancer increases the translucency of the dental restoration in the sintering process. In contrast, an opacity enhancer increases the opacity of the dental restoration in the sintering process. The reciprocal (mutual) property to translucency is opacity (impermeability to light).

The method allows the number of print heads of a production apparatus to be reduced without restricting the degrees of freedom of color, translucency, opacity and hardness in the dental restoration. This allows the selective material application of the slurry, the selective coloring and the selective adjustment of opacity, translucency and hardness to be carried out in separate process steps. In this way, lifelike dental restorations can be produced.

In a technically advantageous embodiment of the method, the translucency enhancer comprises oxides of the elements of yttrium, lanthanum, ytterbium, neodymium and/or europium. This achieves the technical advantage, for example, that particularly suitable translucency enhancers are used.

In a further technically advantageous embodiment of the method, the opacity enhancer comprises oxides of the following elements, such as aluminum and/or silicon. This achieves the technical advantage, for example, that particularly suitable opacity enhancers are used.

In a further technically advantageous embodiment of the method, the one or more layers are dried prior to the jet-printing of the translucency enhancer or opacity enhancer. This achieves the technical advantage, for example, of reducing run-off of the translucency enhancer or opacity enhancer.

In a further technically advantageous embodiment of the method, the slurry has an yttrium content of less than 3 mol %. This achieves the technical advantage, for example, that an opaque slurry is used which can be made translucent by means of a translucency enhancer. In this case, an opacity enhancer can be dispensed with.

In a further technically advantageous embodiment of the method, the slurry has an yttrium content of between 3.5 and 4.5 mol %. This achieves the technical advantage, for example, that both the translucency and the opacity of the slurry can be changed.

In a further technically advantageous embodiment of the method, a coloring solution is jet-printed onto the one or more layers. This achieves the technical advantage, for example, that the layers can be colored in a location-selective manner.

In a further technically advantageous embodiment of the method, the coloring solution is doped with a translucency enhancer or opacity enhancer. This achieves the technical advantage, for example, that translucency and opacity can be adjusted simultaneously during coloring.

In a further technically advantageous embodiment of the method, the produced dental restoration is subjected to a drying and/or debinding step prior to a sintering process. This drying and/or debinding step can take place in a separate thermal process or is an upstream process step in the sintering process. Drying is usually carried out at temperatures of 25° C. to 200° C., preferably 30° C. to 180° C. and particularly preferably 40°° C. to 150° C. In addition, the humidity can be regulated between 10 and 90%, preferably 15 to 85% and particularly preferably 20 to 80%. Debinding is usually carried out at temperatures of 50° C. to 600° C., preferably between 100° C. and 600° C. and particularly preferably at 200° C. to 600° C. The heating rates are between 0.1 and 10 K/min, preferably between 0.2 and 10 K/min and particularly preferably between 0.5 and 10 K/min.

In a further technically advantageous embodiment of the method, the dental restoration is sintered in a sintering furnace. This achieves the technical advantage, for example, that a dental restoration with high strength can be produced. According to a second aspect, the technical task is solved by a production apparatus for producing a dental restoration by jet-printing, comprising a first print head for jet-printing one or more layers of the dental restoration by means of a ceramic slurry; and a second print head for jet-printing a translucency enhancer or opacity enhancer onto the one or more layers. The production apparatus achieves the same technical advantages as the method according to the first aspect.

In a technically advantageous embodiment of the production apparatus, the production apparatus comprises a receiving container for the translucency enhancer and/or opacity enhancer. The respective receiving container can be replaceable. This achieves the technical advantage, for example, that the translucency enhancer and/or opacity enhancer is stored in the production apparatus and can be replaced easily.

In a further technically advantageous embodiment of the production apparatus, the production apparatus comprises a drying device for drying the one or more layers. This achieves the technical advantage, for example, that a run-off of the translucency enhancer and/or opacity enhancer can be reduced.

In a further technically advantageous embodiment of the production apparatus, the drying device comprises a blower and/or an infrared emitter. This achieves the technical advantage, for example, that the layers can be dried quickly and free of cracks.

In a further technically advantageous embodiment of the production apparatus, the production apparatus comprises a dithering module for calculating intermediate translucency values. This achieves the technical advantage, for example, that the dental restoration can be produced with finely tuned translucency values.

In a further technically advantageous embodiment of the production apparatus, the dithering module is adapted to use different two-dimensional dithering patterns in successive layers of the dental restoration. This has the technical advantage, for example, of preventing the formation of stripe or moiré patterns in the dental restoration.

According to a third aspect, the technical task is solved by a jet-printing system, comprising a production apparatus for producing a dental restoration by jet-printing; a ceramic slurry for printing one or more layers; and a translucency enhancer and/or an opacity enhancer for jet-printing onto the one or more layers. The jet-printing system achieves the same technical advantages as the method according to the first aspect.

In a technically advantageous embodiment of the jet-printing system the ceramic slurry, the translucency enhancer and/or the opacity enhancer is stored in a container. This achieves the technical advantage, for example, that the ceramic slurry, the translucency enhancer and/or the opacity enhancer can be stored reliably.

In a further technically advantageous embodiment of the jet-printing system the container is replaceable. This achieves the technical advantage, for example, that the solutions can be exchanged easily.

### BRIEF DESCRIPTION OF THE DRAWINGS

Exemplary embodiments of the invention are shown in the drawings and are described in more detail below, in which:

FIG. 1 shows a representation of different areas of an incisor;

FIG. 2 shows a schematic representation of a dental restoration;

FIG. 3 shows a schematic representation of a production apparatus for producing a dental restoration;

FIG. 4 shows a schematic representation of an increase of the translucency;

FIG. 5 shows a schematic representation of an increase of the translucency or an increase of the opacity; and

FIG. 6 shows a block diagram of a method for producing a dental restoration.

### DETAILED DESCRIPTION

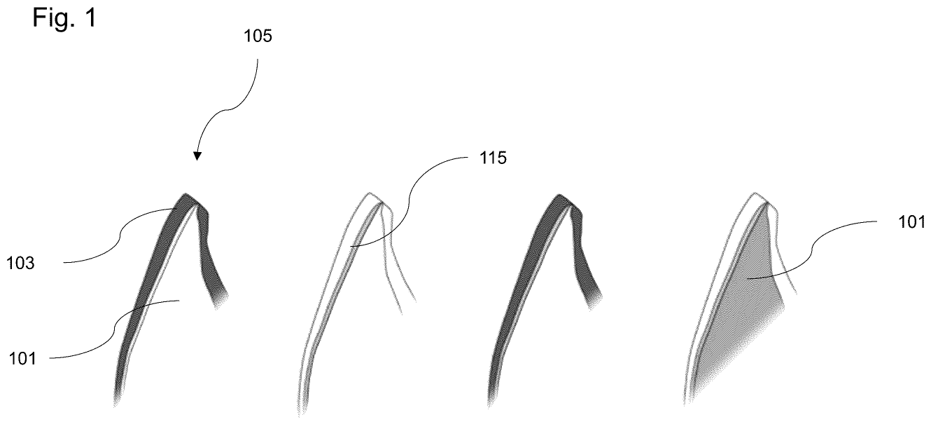

FIG. 1 shows a representation of different areas of a tooth 105. The tooth 105 comprises an inner dentin core 101 and an outer enamel 103. The opaque, i.e. non-transparent, dentin core 101 is responsible for the basic coloring of the tooth 105. The dentin core 101 shines through the enamel 103 of the tooth edge. The enamel 103, on the other hand, is often transparent, i.e. translucent. Translucency is the partial light transmission of a body. In order to make a dental restoration look as lifelike as possible, this structure of the tooth 105 is also used in artificial dental restorations. For this purpose, materials with different optical properties are used in the production of the dental restoration.

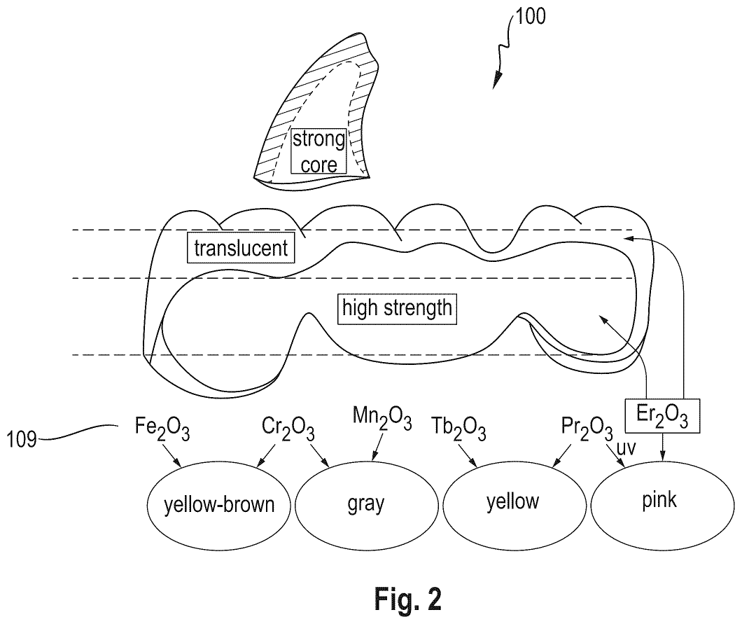

FIG. 2 shows a schematic representation of a dental restoration 100. The dental restoration 100 serves as a dental prosthesis and is formed, for example, by a bridge, a crown, a full or partial prosthesis. The dental restoration 100 is built up, for example, by means of different ceramic slurries 109, which comprise Fe₂O₃, Cr₂O₃, Mn₂O₃, Tb₂O₃, Pr₂O₃, Er₂O₃, CeO₂, NiO, TiO₂ and Co₃O₄. For this purpose, these slurries 109 are used selectively in the respective areas during the spatial production of the dental restoration 100 by means of a jet-printing process.

The dental restoration 100 is built up by successive layers that are printed on top of each other. The ceramic powders of the slurries 109 can already be provided in predetermined translucencies. Mixing these slurries 109 results in the target translucency of the dental restoration 100 in the respective spatial area.

After the selective material application of a layer of the slurry 109 using the jet-printing process, this layer is dried free of cracks by evaporating the water or the solvent as a binder. What remains is a porous white body layer with a layer thickness of 1 μm to 50 μm and a density of at least 2.5 g/cm³. This process is repeated until the entire dental restoration 100 is built up spatially layer by layer.

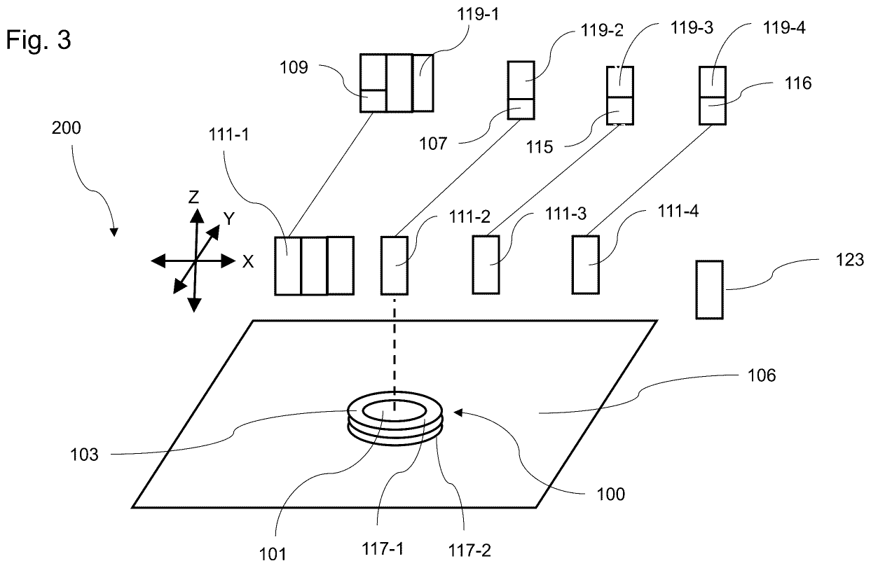

FIG. 3 shows a schematic representation of a production apparatus 200 for producing a dental restoration 100 by jet-printing (inkjet-printing) of aqueous or solvent-based slurries 109. In order to additively produce ceramic, dental multi-material and multi-color restorations 100 by jet-printing, base slurries 109 are processed.

The production apparatus 200 comprises a plurality of receiving containers 119-1 in which the base slurries 109 with different optical properties are arranged. Furthermore, the production apparatus 200 comprises at least one receiving container 119-2 in which a translucency enhancer or an opacity enhancer is stored. In addition, the production apparatus 200 comprises a receiving container 119-3 for receiving a coloring solution 115 and a receiving container 119-4 for receiving a fixing solution 116. The production apparatus for producing the dental restoration by jet-printing; the ceramic slurry; the translucency enhancer and/or an opacity enhancer for jet-printing onto the one or more layers form together a jet-printing system.

The ceramic slurries 109 are each applied drop by drop in several layers 117-1, . . . , 117-n by means of an assigned print head 111-1 in order to spatially build up the dental restoration 100 layer by layer. The print head and the build platform 106 for applying the slurry are movable relative to each other in 3 spatial axes (XYZ) so that the slurry 109 can be printed at any position. Slurries 109 with a drop volume of typically 10 to 100 pL are used for the selective material application. Electrically controlled piezo elements, for example, are used to eject the droplets.

The use of solvent-based or aqueous ceramic slurries and the evaporation of the solvent or water after applying a coating by drying eliminates the time-consuming debinding process that would be required if polymers were used as binders.

For the application of the translucency enhancer or opacity enhancer 107, a further print head 111-2 is used, by means of which the translucency enhancer or opacity enhancer 107 can be jet-printed onto one or more layers 117-1, . . . , 117-n. The print head 111-2 is also movable in 3 spatial axes (XYZ) relative to the build platform 106, so that the translucency enhancers or opacity enhancers 107 can be printed at any position.

A further print head 111-3 is used for applying the coloring solution 115, by means of which the coloring solution 115 can be jet-printed onto one or more layers 117-1, . . . , 117-n. The print head 111-3 is also movable in three spatial axes (XYZ) relative to the build platform 106, so that the coloring solution can be printed at any position.

For the application of the fixing solution 116, which serves to fix the coloring solution 115 or the translucency enhancer or opacity enhancer 107, a further print head 111-4 is used, by means of which the fixing solution 116 can be jet-printed onto one or more layers 117-1, . . . , 117-n. The print head 111-4 is also movable in 3 spatial axes (XYZ) relative to the build platform 106, so that the coloring solution can be printed at any position.

The print heads 111-1, 111-2, 111-3 and 111-4 can be independently controllable or integrated in a common print module.

Of course, it is also possible that the print heads 111 are stationary and the build platform 106 with the printed dental restoration 100 and the most recently printed layers 117 moves in 3 directions (XYZ) relative to the print heads 111 in order to be able to print slurries 109, translucency enhancers or opacity enhancers 107, a coloring solution 115 or fixing solution 116 at any position.

A drying device 123 is provided for drying the aqueous material, with which the layers 117-1, . . . , 117-n and the translucency enhancer or opacity enhancer 107 can be dried free of cracks. The drying device 123 is formed, for example, by a blower and/or an infrared emitter.

The production apparatus 200 provides a reduced number of pre-colored and yttrium-doped neutral base slurries 109 in order to minimize the number of print heads 111-1 and 111-2 and still achieve an aesthetic and functional result of the dental restoration 100. A subtractive color system is used, which spans a limited dental color space (dental color gamut). The color mixture, the three-dimensional halftoning or dithering of these pre-colored slurries 109 in various ratios is then created in the special dental color space (gamut), which covers the common tooth colors, but not all colors.

During production, translucency enhancers or opacity enhancers 107 are selectively applied onto a layer 117-1, . . . , 117-n via a 3D dithering algorithm, which is executed by a dithering module 113. For this purpose, the dithering module 113 comprises a processor for executing the 3D dithering algorithm and a digital data memory for storing the calculated mixing ratios and the dithering algorithm. The processor comprises any hardware system, component, or mechanism that processes data, signals, or other information. A processor may comprise a system with a central processing unit (CPU), multiple data processing units (MPU), dedicated electrical circuitry to achieve functionality, or other systems. The data memory may comprise hard disks, flash memory cards, random access memories (RAM) or read-only memories (ROM).

During dithering, the translucency enhancer or opacity enhancer 107 is selectively applied in a certain ratio and two-dimensional print pattern in the printing plane using the jet-printing process. The 3D dithering algorithm also calculates that non-identical two-dimensional dithering patterns are applied in multiple layers 117-1, . . . , 117-n on top of each other. This prevents optical artifacts such as stripe or moiré patterns on vertical surfaces. The color mixing, three-dimensional halftoning or dithering of these pre-colored slurries 109 in various ratios is created within the special dental color space (gamut), which covers the common tooth colors, but not all general colors.

When using highly viscous slurries 109 with a viscosity of more than 100 mPas with a high degree of filling, special requirements are placed on the print head 111-1 and its fluid system. The print head 111-1 is compatible with the binder used and/or the carrier material of the slurry 109, such as water in the case of aqueous slurries 109, in order to prevent corrosion.

After the selective material application of a layer 117-1, . . . , 117-n of the slurry 109 by means of the jet-printing process, this layer 117-1, . . . , 117-n is dried free of cracks by evaporating the water or the solvent as a binder. The ceramic powder of the slurry 109 can already be colored in a color, for example a base color or tooth color. What remains is a porous white body layer with a layer thickness of 1 μm to 50 μm and a density of at least 3.0 g/cm³. This process is repeated until the entire dental restoration 100 is built up spatially layer by layer.

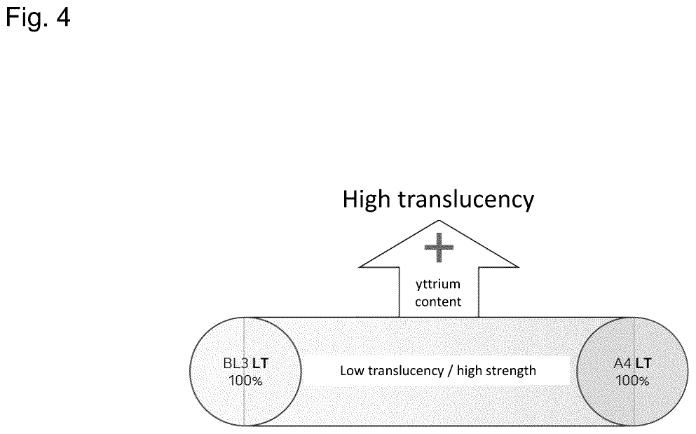

FIG. 4 shows a schematic representation of an increase in translucency. The dental restoration 100 is built up in layers from a slurry 109, which has a low translucency and a high strength. Therefore, the opacity of the slurry 109 is high. For example, a slurry with an yttrium content of 3 mol % yttrium—3Y-TZP is used for this purpose. Starting from this slurry 109, the translucency is increased location-selectively by jet-printing the translucency enhancer. The translucency can be adjusted in the range from CR value 90% to 50% by position-selective printing of the translucency enhancer.

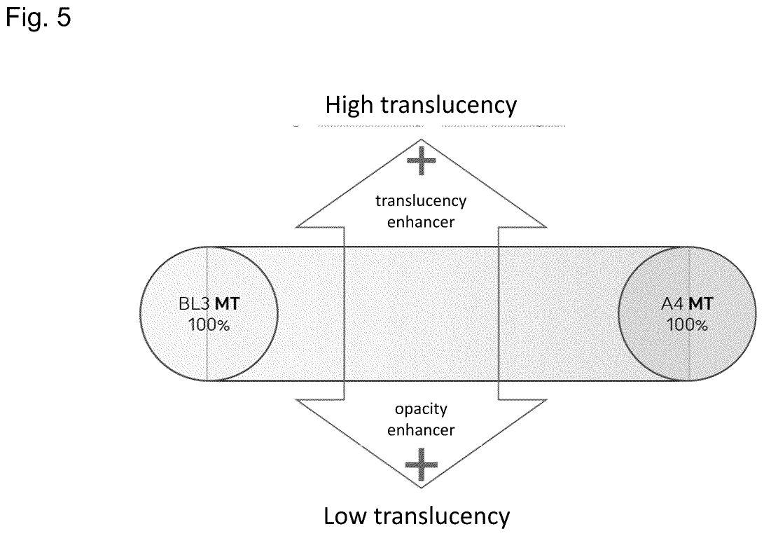

FIG. 5 shows a schematic representation of an increase in translucency or opacity. The dental restoration 100 is built up in layers from a slurry 109 that has a medium translucency or opacity. For example, a slurry with an yttrium content of 4 mol % yttrium—4Y-TZP is used for this purpose.

Starting from this slurry 109, the translucency is increased position-selectively by jet-printing the translucency enhancer 107 or the opacity by jet-printing an opacity enhancer 107. In this way, the translucency or opacity properties of the slurry 109 can be adapted in a targeted manner.

In addition to the local selective adjustment of translucency, opacity and hardness during the layer build-up of the dental restoration 100, it can also be selectively colored locally using coloring solutions 115. This is advantageous if only a single base slurry with medium translucency in a basic color is used. The coloring solutions 115 may already be doped with the appropriate rare earths in order to further reduce the required number of print heads 111 without restricting the degrees of freedom of color, translucency, opacity and hardness.

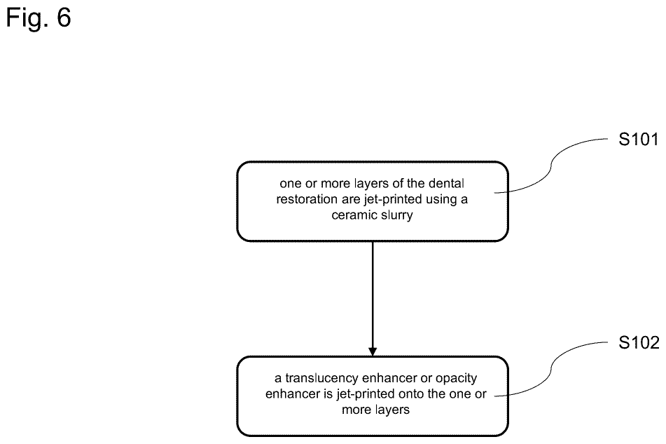

FIG. 6 shows a block diagram of a method of producing a dental restoration. In step S101, one or more layers 117-1, . . . , 117-n of the dental restoration 100 are jet-printed using the ceramic slurry 109. In step S102, a translucency enhancer or opacity enhancer 107 is jet-printed onto the one or more layers 117-1, . . . , 117-n. Here, a single slurry 109 in a basic coloring and a medium translucency, e.g. with an yttrium content of 4 mol % (4Y-TZP), can be used for the additive material application of the dental restoration 100. The selective coloring is achieved by jet-printing of coloring solutions 115. Slurries with discrete particles in the nano (5 nm-100 nm) or submicron range (100 nm-1 μm) or ionic solutions with a high concentration can be used.

The selective adjustment of opacity, translucency and hardness is achieved by the selective application of ionic solutions with variable components of yttrium, lanthanum, ytterbium and other rare earths. For example, Y³⁺, La³⁺, Yb³⁺, Nd³⁺ and Eu³⁺ are used as translucency enhancers and Al³⁺, Si as opacity enhancers.

Typically, the opaque dentin core is responsible for the color and the translucent edge for the vibrancy of the dental restoration 100. With a suitably doped translucency enhancer, the yttrium content can be increased from 3Y to 5Y, for example. This allows different opacity values to be achieved in the dental restoration 100 in a location-selective manner, such as opaque spatial areas for the dentin core and translucent spatial areas for the edge.

Highly esthetic and functional dental restorations 100 can be produced by mixing two pre-colored slurries 109 with light and dark tooth colors of medium translucency and opacity and selectively adding a translucency enhancer or opacity enhancer. For example, two high-strength and opaque slurries can be mixed for the dentin core and a translucency enhancer can be added in corresponding areas for a less solid, translucent incisal edge. In this case, only 4 print heads are required:
1 Support material
2 3Y-TZP slurry—light (opaque, high-strength)
3 3Y-TZP slurry—dark (opaque, high-strength)
4 Yttrium solution (for local control of translucency and strength)

Alternatively, the dental restoration could be built up with a medium-translucent slurry 109 to which a translucency enhancer or an opacity enhancer (opaquer liquid) is selectively added.

The selective addition of the translucency enhancer or opacity enhancer can take place on the wet jet-printed layer 117-1, . . . , 117-n (wet on wet) or on the dry jet-printed layer 117-1, . . . , 117-n (wet on dry). The chemical conversion takes place during the subsequent sintering process of the selectively doped green body. This produces the desired optical properties.

All the features explained and shown in connection with individual embodiments of the invention can be provided in different combinations in the subject-matter according to the invention in order to simultaneously realize their advantageous effects.

All method steps can be implemented by devices that are suitable for executing the respective method step. All functions performed by the features of the subject-matter can be a method step of a method.

The scope of protection of the present invention is given by the claims and is not limited by the features explained in the description or shown in the figures.

### LIST OF REFERENCE SIGNS

100 Dental restoration
101 Dentin core
103 Enamel
105 Tooth
106 Build platform
107 Translucency enhancer/opacity enhancer
109 Slurry
111 Print head
113 Dithering module
115 Coloring solution
116 Fixing solution
117 Layer
119 Receiving container
121 Application device
123 Drying device

1. A method of producing a dental restoration by jet-printing, comprising
jet-printing one or more layers of the dental restoration using a ceramic slurry;
jet-printing a translucency enhancer or opacity enhancer onto the one or more layers.

2. The method according to claim 1,
wherein the translucency enhancer comprises oxides of the elements of yttrium, lanthanum, ytterbium, neodymium and/or europium.

3. The method according to claim 1,
wherein the opacity enhancer comprises aluminum and/or silicon.

4. The method according to claim 1,
wherein the one or more layers are dried prior to the jet-printing of the translucency enhancer or opacity enhancer.

5. The method according to claim 1,
wherein the slurry has an yttrium content of less than 3 mol %.

6. The method according to claim 1,
wherein the slurry has an yttrium content of between 3.5 and 4.5 mol %.

7. The method according to claim 1,
wherein a coloring solution is jet-printed onto the one or more layers.

8. The method according to claim 1,
wherein the coloring solution is doped with a translucency enhancer or opacity enhancer.

9. The method according to claim 1,
wherein the produced dental restoration is subjected to a drying and/or debinding step prior to a sintering process.

10. A production apparatus for producing a dental restoration by jet-printing, comprising:
a first print head for jet-printing one or more layers of the dental restoration using a ceramic slurry; and
a second print head for jet-printing a translucency enhancer or opacity enhancer onto the one or more layers.

11. The production apparatus according to claim 10,
wherein the production apparatus comprises a receiving container for the translucency enhancer and/or opacity enhancer.

12. The production apparatus according to claim 10,
wherein the production apparatus comprises a drying device for drying the one or more layers.

13. The production apparatus according to claim 10,
wherein the drying device comprises a blower and/or an infrared emitter.

14. The production apparatus according to claim 10,
wherein the production apparatus comprises a dithering module for calculating intermediate translucency values.

15. The production apparatus according to claim 10,
wherein the dithering module is adapted to use different two-dimensional dithering patterns in successive layers of the dental restoration.

16. A jet-printing system, comprising:
a production apparatus for producing a dental restoration by jet-printing;
a ceramic slurry for printing one or more layers; and
a translucency enhancer and/or an opacity enhancer for jet-printing onto the one or more layers.

17. The jet-printing system according to claim 16,
wherein the ceramic slurry, the translucency enhancer and/or the opacity enhancer is/are stored in a container.

18. The jet-printing system according to claim 17,
wherein the container is replaceable.
